(19) United States
(12) Patent Application Publication
(10) Pub. No.: US 2008/0032290 A1
(43) Pub. Date: Feb. 7, 2008

(54) Nanopore flow cells

(76) Inventor: James E. Young, La Honda, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/498,566

(22) Filed: Aug. 3, 2006

(51) Int. Cl. C12Q 1/68; C12M 3/00

(52) U.S. Cl. 435/6; 435/287.2; 977/924

(57) ABSTRACT

Systems and methods for nanopore flow cells are provided.

### BACKGROUND

Determining the nucleotide sequence of DNA and RNA in a rapid manner is a major goal of researchers in biotechnology, especially for projects seeking to obtain the sequence of entire genomes of organisms. In addition, rapidly determining the sequence of a nucleic acid molecule is important for identifying genetic mutations and polymorphisms in individuals and populations of individuals.

Nanopore sequencing is one method of rapidly determining the sequence of nucleic acid molecules. Nanopore sequencing is based on the property of physically sensing the individual nucleotides (or physical changes in the environment of the nucleotides (i.e., electric current)) within an individual polynucleotide (e.g., DNA and RNA) as it traverses through a nanopore aperture. In principle, the sequence of a polynucleotide can be determined from a single molecule. However, in practice, it is preferred that a polynucleotide sequence be determined from a statistical average of data obtained from multiple passages of the same molecule or the passage of multiple molecules having the same polynucleotide sequence. The use of membrane channels to characterize polynucleotides as the molecules pass through the small ion channels has been studied by Kasianowicz et al. (Proc. Natl. Acad. Sci. USA. 93:13770-3, 1996, incorporate herein by reference) by using an electric field to force single stranded RNA and DNA molecules through a 2.6 nanometer diameter nanopore aperture (i.e., ion channel) in a lipid bilayer membrane. The diameter of the nanopore aperture permitted only a single strand of a polynucleotide to traverse the nanopore aperture at any given time. As the polynucleotide traversed the nanopore aperture, the polynucleotide partially blocked the nanopore aperture, resulting in a transient decrease of ionic current. Since the length of the decrease in current is directly proportional to the length of the polynucleotide, Kasianowicz et al. were able to experimentally determine lengths of polynucleotides by measuring changes in the ionic current.

Baldarelli et al. (U.S. Pat. No. 6,015,714) and Church et al. (U.S. Pat. No. 5,795,782) describe the use of nanopores to characterize polynucleotides including DNA and RNA molecules on a monomer by monomer basis. In particular, Baldarelli et al. characterized and sequenced the polynucleotides by passing a polynucleotide through the nanopore aperture. The nanopore aperture is imbedded in a structure or an interface that separates two media. As the polynucleotide passes through the nanopore aperture, the polynucleotide alters an ionic current by blocking the nanopore aperture. As the individual nucleotides pass through the nanopore aperture, each base/nucleotide alters the ionic current in a manner which allows the identification of the nucleotide transiently blocking the nanopore aperture, thereby allowing one to characterize the nucleotide composition of the polynucleotide and perhaps determine the nucleotide sequence of the polynucleotide.

### SUMMARY

Systems and methods for nanopore flow cells are provided. One such nanopore analysis system, among others, includes a nanopore flow cell including a cell reservoir, at least one fluid flow channel, an electrode, and a nanopore aperture. The cell reservoir is in fluid communication with the fluid flow channel. The nanopore aperture is in fluid communication with the cell reservoir. The cell reservoir is in fluid communication with the electrode. The nanopore flow cell also includes a first structure having the nanopore aperture; a second structure adjacent the first structure, the second structure including a first opening that defines a portion of the cell reservoir and a second opening that defines a portion of the fluid flow channel; and a third structure adjacent the second structure, the third structure having the electrode disposed thereon and an opening for the fluid flow channel. A portion of the cell reservoir is defined on a first side by the first structure and another portion of the cell reservoir is defined on a second side by the third structure. A portion of the fluid flow channel is defined on a first side by the first structure. A fluid can flow through the openings of the fluid flow channel into the fluid flow channels and into the cell reservoir.

One such method for analyzing a polynucleotide, among others, includes providing a nanopore analysis system as described above; introducing a target polynucleotide to the cell reservoir via the fluid flow channel; applying a voltage gradient to the nanopore analysis system; translocating the target polynucleotide through the nanopore aperture; and monitoring the signal corresponding to the movement of the target polynucleotide with respect to the nanopore aperture.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

Reference is now made to the following drawings. Note that the components in the drawings are not necessarily to scale.

FIG. 1 is a schematic of an embodiment of a nanopore analysis system.

FIGS. 2A and 2B are diagrams of representative nanopore devices that can be used in the nanopore analysis system of FIG. 1.

FIGS. 3A and 3B are diagrams of representative nanopore flow cells and can be used in the nanopore analysis system of FIG. 1.

FIGS. 4A and 4B are diagrams of representative nanopore flow cells and can be used in the nanopore analysis system of FIG. 1.

FIG. 5 illustrates a perspective view of a structure that can be used in the nanopore analysis system of FIGS. 3A and 3B and/or FIGS. 4A and 4B.

### DETAILED DESCRIPTION

As will be described in greater detail here, nanopore analysis systems incorporating nanopore flow cell systems, are provided. By way of example, some embodiments provide for a plurality of structures that include openings for fluid to flow once the structures are secured to one another. The openings can include, but are not limited to, fluid flow channels, reservoirs, and the like. The fluid flow channels can be used to introduce fluids to the reservoirs from a fluid source within or outside of the nanopore analysis system. In one embodiment, the reservoir is in fluid communication with a nanopore aperture, where molecules (e.g., nucleotides, peptides, and the like) can, under proper conditions, interact with the nanopore aperture. In another embodiment, the fluid flow channels can be positioned so that the reservoir is filled from the bottom of the reservoir, which reduces the probability of air bubbles blocking a part or all of the nanopore aperture.

The nanopore flow cell systems can be used in nanopore sequencing of polynucleotides, which has been described in U.S. Pat. No. 5,795,782 to Church et al.; and U.S. Pat. No. 6,015,714 to Baldarelli et al., the teachings of which are both incorporated herein by reference. In general, nanopore sequencing involves the use of two separate pools of a medium and an interface between the pools. The interface between the pools is capable of interacting sequentially with the individual monomer residues of a polynucleotide present in one of the pools. Interface dependent measurements are continued over time, as individual monomer residues of the polynucleotide interact sequentially with the interface, yielding data suitable to infer a monomer-dependent characteristic of the polynucleotide. The monomer-dependent characterization achieved by nanopore sequencing may include identifying physical characteristics such as, but not limited to, the number and composition of monomers that make up each individual polynucleotide, in sequential order.

The term “sequencing” as used herein means determining the sequential order of nucleotides in a polynucleotide molecule. Sequencing, as used herein, includes in the scope of its definition, determining the nucleotide sequence of a polynucleotide in a de novo manner in which the sequence was previously unknown. Sequencing, as used herein, also includes in the scope of its definition, determining the nucleotide sequence of a polynucleotide wherein the sequence was previously known. Sequencing polynucleotides, the sequences of which were previously known, may be used to identify a polynucleotide, to confirm a polynucleotide, or to search for polymorphisms and genetic mutations.

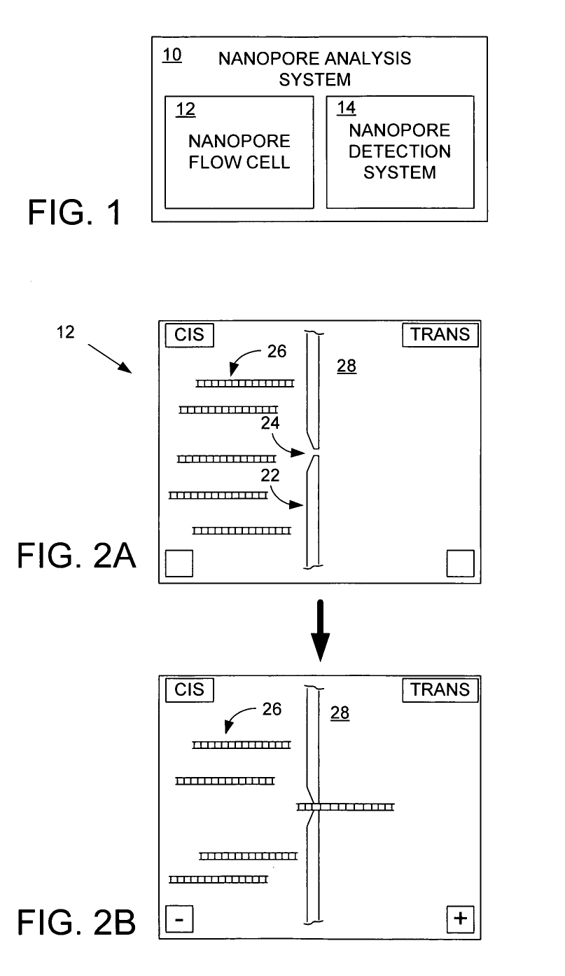

FIG. 1 illustrates a representative embodiment of a nanopore analysis system 10 that can be used in nanopore sequencing. The nanopore analysis system 10 includes, but is not limited to, a nanopore flow cell 12 (also referred to as nanopore device), and a nanopore detection system 14. The nanopore flow cell 12 and the nanopore detection system 14 are communicatively coupled so that data regarding the target polynucleotide can be measured.

The nanopore detection system 14 includes, but is not limited to, electronic equipment capable of measuring characteristics of the polynucleotide as it interacts with the nanopore aperture, a computer system capable of controlling the measurement of the characteristics and storing the corresponding data, control equipment capable of controlling the conditions of the nanopore flow cell 12, control equipment capable controlling the flow of fluids into and out of the nanopore flow cell 12, and components that are included in the nanopore flow cell 12 that are used to perform the measurements as described below.

The nanopore detection system 14 can measure characteristics such as, but not limited to, the amplitude or duration of individual conductance or electron tunneling current changes across the nanopore aperture. Such changes can identify the monomers in sequence, as each monomer has a characteristic conductance change signature. For instance, the volume, shape, or charges on each monomer can affect conductance in a characteristic way. Likewise, the size of the entire polynucleotide can be determined by observing the length of time (duration) that monomer-dependent conductance changes occur. Alternatively, the number of nucleotides in a polynucleotide (also a measure of size) can be determined as a function of the number of nucleotide-dependent conductance changes for a given nucleic acid traversing the nanopore aperture. The number of nucleotides may not correspond exactly to the number of conductance changes, because there may be more than one conductance level change as each nucleotide of the nucleic acid passes sequentially through the nanopore aperture. However, there is a proportional relationship between the two values that can be determined by preparing a standard with a polynucleotide having a known sequence.

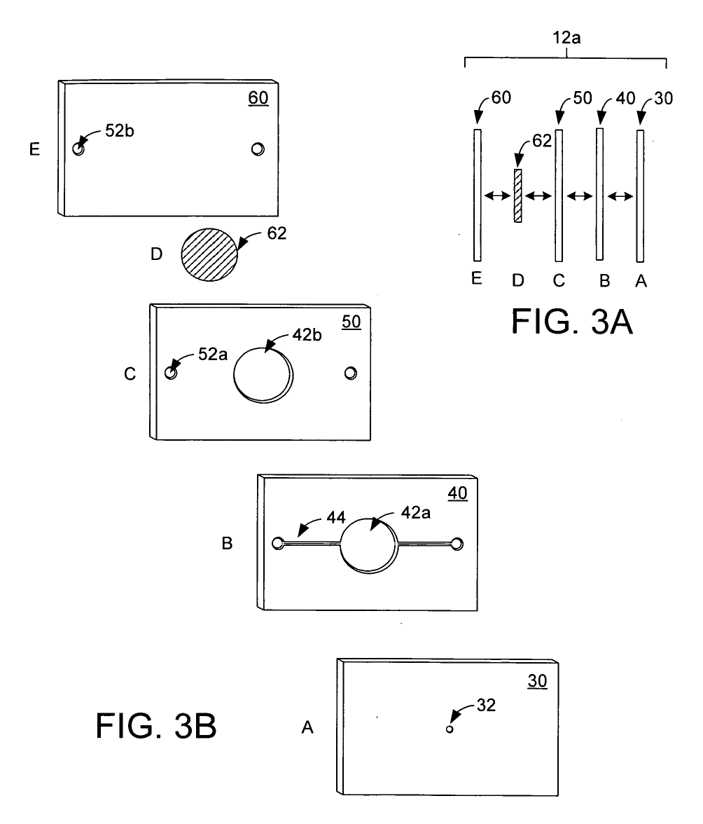

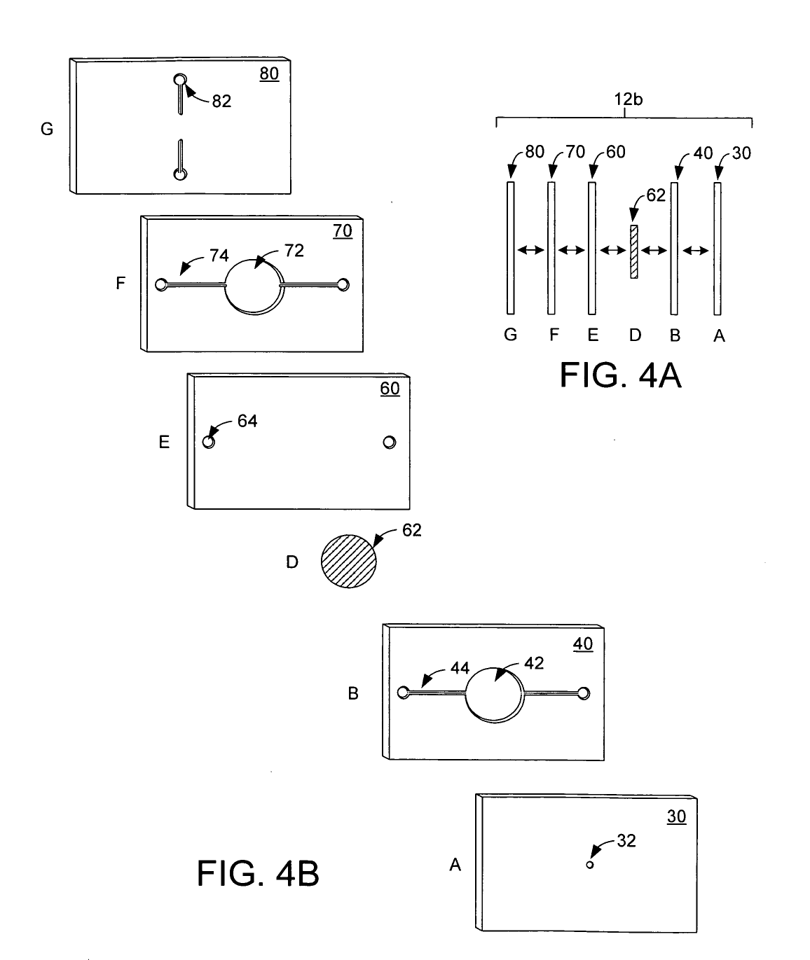

FIGS. 2A and 2B illustrate representative embodiments of the nanopore device 12. The nanopore flow cell 12 includes, but is not limited to, a structure 22 (i.e., the first structure 30 in FIGS. 3A through 4B) that separates two independent adjacent pools of a medium 28 (i.e., one pool being in the reservoir 42a and 42b in FIG. 3B). The two adjacent pools are located on the cis side and the trans side of the nanopore flow cell 12. The structure 22 includes, but is not limited to, at least one nanopore aperture 24 (i.e., nanopore aperture 32 in FIGS. 3A through 4B) so dimensioned as to allow sequential monomer-by-monomer translocation (i.e., passage) from one pool to another of only one polynucleotide at a time, and detection components that can be used to perform measurements of the target polynucleotide.

Exemplary detection components have been described in WO 00/79257 and can include, but are not limited to, electrodes directly associated with the structure 22 at or near the pore aperture 24, and electrodes placed within the cis and trans pools. The electrodes may be capable of, but are not limited to, detecting ionic current differences across the two pools or electron tunneling currents across the pore aperture.

As the polynucleotide 26 translocates through or passes sufficiently close to the nanopore aperture 24, measurements (e.g., ionic flow measurements, including measuring duration or amplitude of ionic flow blockage) can be taken by the nanopore detection system 14 as each of the nucleotide monomers of the polynucleotide passes through or sufficiently close to the nanopore aperture 24. The measurements can be used to identify the sequence and length of the polynucleotide.

The medium 28 disposed in the pools on either side of the substrate 22 may be any fluid that permits adequate polynucleotide mobility for substrate interaction.

The target polynucleotide being characterized may remain in its original pool, or it may cross the nanopore aperture 24 into the other pool. In either situation, the target polynucleotide moves in relation to the nanopore aperture 24, individual nucleotides interact sequentially with the nanopore aperture 24 to induce a change in the conductance of the nanopore aperture 24. The nanopore aperture 24 can be traversed either by a polynucleotide translocation through the nanopore aperture 24 so that the polynucleotide passes from one of the pools into the other, or by the polynucleotide traversing across the nanopore aperture 24 without crossing into the other pool. In the latter situation, the polynucleotide is close enough to the nanopore aperture 24 for its nucleotides to interact with the nanopore aperture 24 passage and bring about the conductance changes, which are indicative of polynucleotide characteristics.

Now having described the nanopore flow cell 12 in general, FIGS. 3A, 3B, 4A, 4B, and 5 describe additional features of the nanopore flow cell 12. Please note that the entire nanopore flow cell 12 is not depicted in FIGS. 3A, 3B, 4A, and 4B, but rather one side of the nanopore flow cell 12. The remaining portions of the nanopore flow cell 12 are generally known to one skilled in the art. In short, once the target molecule translocates through the nanopore aperture, the fluid on the trans side of the nanopore flow cell 12 is discarded or additionally treated.

FIG. 3A is a cross-sectional view of a portion (i.e., the cis side of the nanopore flow cell 12a) of the nanopore flow cell 12a, while FIG. 3B illustrates a perspective view of the same portion of the nanopore flow cell 12a as shown in FIG. 3A. The nanopore flow cell 12a includes, but is not limited to, a nanopore aperture 32, a cell reservoir 42a and 42b, fluid flow channels 44, and an electrode 62 (e.g., a silver/silver chloride electrode or the like). The nanopore aperture 32 is in fluid communication with the cell reservoir 42a and 42b. The cell reservoir 42a and 42b is in fluid communication with the fluid flow channels 44. The cell reservoir 42a and 42b is in fluid communication with the electrode 62.

The nanopore flow cell 12a includes, but is not limited to, a first structure 30, a second structure 40, a spacer structure 50, and a third structure 60. The first structure 30 includes the nanopore aperture 32 (e.g., about 2 to 5 nanometers in diameter). The second structure 40 is adjacent the first structure 30. The second structure 40 includes a first opening (42a) that defines a portion of the cell reservoir (42a and 42b) and a second opening (44) that defines a portion of the fluid flow channel 44. The spacer structure 50 is disposed between the second structure 40 and a third structure 60. The spacer structure 50 includes an opening 52a in fluid communication with the fluid flow channel 44 and an opening (42b) for the cell reservoir 42a and 42b. The electrode 62 is disposed on the surface of the third structure 60 and in-line (e.g., all or a portion of the electrode 62 is exposed to the openings of the cell reservoir 42a and 42b) with the cell reservoir 42a and 42b. In addition, the third structure 60 includes an opening 52b for the fluid flow channel 44. In another embodiment, the nanopore flow cell 12a does not include the spacer structure 50 and the third structure 60 is adjacent the second structure 40.

In other words, the first structure 30, the second structure 40, the spacer structure 50, and the third structure 60, are aligned and secured against one another to form a part of the nanopore flow cell 12a. The openings form the flow channels and reservoirs of the nanopore flow cells in which fluids and samples flow. The first structure 30, the second structure 40, the spacer structure 50, and the third structure 60, can be secured against one another by physical (e.g., mechanical (e.g., screws), heat and/or pressure, and the like) and/or chemical (e.g., adhesives and the like) securing mechanisms.

In particular, a portion of the cell reservoir 42a and 42b is defined on a first side by the first structure 30, and another portion of the cell reservoir is defined on a second side by the third structure 60 and the electrode 62. In addition, the spacer structure 50 defines a portion of the cell reservoir 42a and 42b.

A portion of the fluid flow channel 44 is defined on a first side by the first structure 30 and on a second side by the spacer structure 50. In addition, the fluid flow channel 44 flows through openings 52a in the spacer structure 50 and the openings 52b of the third structure 60 from an appropriate fluid or sample introduction system (not shown). A sample or other fluid can flow into and/or out of the cell reservoir 42a and 42b via one or more of the openings 52b and 52a and one or more of the fluid flow channels 44. The openings 52a and 52b and openings for the fluid flow channels 44 can be reversibly opened and closed to facilitate proper flow into and out of the cell reservoir 42a and 42b. In another embodiment, some of the openings 52a and 52b may not be present to facilitate proper flow into and out of the cell reservoir 42a and 42b.

The nanopore aperture 32 can be dimensioned so that only a single stranded polynucleotide can translocate through the nanopore aperture 32 at a time or so that a double or single stranded polynucletide can translocate through the nanopore aperture 32. The nanopore aperture 32 can have a diameter of about 3 to 5 nanometers (for analysis of single or double stranded polynucleotides) and from about 2 to 4 nanometers (for analysis of single stranded polynucleotides).

The first structure 30, the second structure 40, the spacer structure 50, and the third structure 60, can each have similar lengths and/or heights (e.g., about 3 to 12 mm). Each structure can have a width (narrowest dimension) of about 50 to 5000 nm. In addition, the width can vary across the structure. Each of the first structure 30, the second structure 40, the spacer structure 50, and the third structure 60, can have a different width. For example the spacer structure 50 may have a minimum width to account for the electrode 62 that is slightly raised on the third structure 60. In another example, the second structure 40 and/or the spacer structure 50 can each have a width to define a specific volume of the cell reservoir 42a and 42b. In this way, the nanopore flow cell 12 can be reconfigured or modified by the addition or removal of substrates to produce nanopore flow cells with different dimensions, fluid flow channels 44, and the like. The widths for each of the first structure 30, the second structure 40, the spacer structure 50, and the third structure 60, can be selected based on the configuration needed for a particular application.

The first structure 30, the second structure 40, the spacer structure 50, and the third structure 60 can be made of materials such as, but not limited to, silicon nitride, silicon oxide, mica, polyimide, stainless steel, polymer, various glasses, ceramics, and the like.

The fluid flow channels 44 and the cell reservoir 42a and 42b can be configured to enhance the operation of the nanopore flow cell 12a. For example, the fluid flow channels 44 and the cell reservoir 42a and 42b can be configured (e.g., one or more of the openings 52a and 52b or the opening to one side of the fluid flow channel 44 is reversibly closed) so that the cell reservoir 42a and 42b is filled from the bottom-up. In other words, one of the fluid flow channels 44 that introduces the sample fluid is positioned at the bottom of the cell reservoir 42a and 42b, for example. By filling the cell reservoir 42a and 42b from the bottom-up, the probability of having an air bubble block the nanopore aperture 32 or a portion thereof is reduced.

FIG. 4A is a cross-sectional view of a portion of the nanopore flow cell 12b, while FIG. 4B illustrates a perspective view of the same portion of the nanopore flow cell 12b as shown in FIG. 4A. The nanopore flow cell 12b includes, but is not limited to, a nanopore aperture 32, a cell reservoir 42, fluid flow channels 44, an electrode 62, a mixing reservoir 72, and mixing fluid flow channels 74. The nanopore aperture 32 is in fluid communication with the cell reservoir 42. The cell reservoir 42 is in fluid communication with the fluid flow channels 44. The cell reservoir 42 is in fluid communication with the electrode 62. The fluid flow channels 44 are in fluid communication with the mixing fluid flow channels 74. The mixing fluid flow channels 74 are in fluid communication with the mixing reservoir 72.

The nanopore flow cell 12b includes, but is not limited to, a first structure 30, a second structure 40, a third structure 60, a first mixing structure 70, and a second mixing structure 80. The first structure 30, the second 40, and the third structure 60, include the same components and may have the same characteristics as those same structures described in FIGS. 3A and 3B. In another embodiment, the nanopore flow cell 12b can include one or more spacer structures.

In addition, the first mixing structure 70 includes a first opening (72) that defines a portion of the mixing cell reservoir 72 and second opening (74) that defines a portion of the mixing fluid flow channel 74. The first mixing structure 70 is adjacent the third structure 60. The second mixing structure 80 includes an opening/channel 82 to flow fluid into the mixing cell reservoir 72, which is in fluid communication with mixing fluid flow channel 74. The second mixing structure 80 is adjacent the first mixing structure 70.

In other words, the first structure 30, the second structure 40, the third structure 60, the first mixing structure 70, and the second mixing structure 80 are aligned and secured against one another to form a part of the nanopore flow cell 12b. The openings form the flow channels and reservoirs of the nanopore flow cells in which fluids and samples flow and are mixed. The first structure 30, the second structure 40, the third structure 60, the first mixing structure 70, and the second mixing structure 80 can be secured against one another by physical (e.g., mechanical (e.g., screws), heat and/or pressure, and the like) and/or chemical (e.g., adhesives and the like) securing mechanisms.

In particular, a portion of the cell reservoir 42 is defined on a first side by the first structure 30, and another portion of the cell reservoir is defined on a second side by the third structure 60 and the electrode 62.

A portion of the fluid flow channel 44 is defined on a first side by the first structure 30 and on a second side by the third structure 60.

A portion of the mixing cell reservoir 72 is defined on a first side by the third structure 60, and another portion of the mixing cell reservoir 72 is defined on a second side by the second mixing structure 80.

A portion of the mixing fluid flow channel 74 is defined on a first side by the third structure 60, and another portion of the mixing cell reservoir 72 is defined on a second side by the second mixing structure 80.

Fluid flows from the openings/channels 82 in the second mixing structure 80 from an appropriate fluid or sample introduction system (not shown) into the mixing cell reservoir 72. Once the fluid is mixed, the fluid can flow out of the mixing cell reservoir 72 through the mixing fluid flow channel 74. Then fluid flows to the fluid flow channel 44 and the cell reservoir 42 via openings 64 in the third structure 60.

The openings 64 and 82 and openings for the fluid flow channels 44 and the mixing fluid flow channels 74, can be reversibly opened and closed to facilitate proper flow into and out of the cell reservoir 42 and/or into and out of the mixing cell reservoir 72. In another embodiment, some of the openings 64 and 82 may not be present to facilitate proper flow into and out of the cell reservoir 42 and/or into and out of the mixing cell reservoir 72.

The first structure 30, the second structure 40, the third structure 60, the first mixing structure 70, and the second mixing structure 80, can each have similar lengths and heights (e.g., about 3 to 12 mm). Each structure can have a width (narrowest dimension) of about 50 to 5000 nm. In addition, the width can vary across the structure. Each of the first structure 30, the second structure 40, the third structure 60, the first mixing structure 70, and the second mixing structure 80, can have a different width. For example the second structure 40 and/or the first mixing structure 70 can each have a width to define a specific volume of the cell reservoir 42 and mixing cell reservoir 72, respectively. In this way, the nanopore flow cell 12b can be reconfigured or modified by the addition or removal of substrates to produce nanopore flow cells with different dimensions, fluid flow channels 44, and the like. The widths for each of the first structure 30, the second structure 40, the third structure 60, the first mixing structure 70, and the second mixing structure 80, can be selected based on the configuration needed for a particular application.

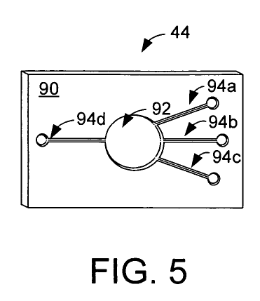

FIG. 5 illustrates a perspective view of a structure 90 that can be used as the second structure 40 and/or the first mixing structure 70. The structure 90 includes a reservoir 92 and fluid flow channels 92a, 92b, 92c, and 92d. One or more of the fluid flow channels 92a, 92b, 92c, and 92d can be used to flow fluid into and/or out of the reservoir 92. For example, three different fluids can be flowed into the reservoir 92 via fluid flow channels 92a, 92b, and 92c, where each channel flows a different fluid. After the fluids mix, react, or otherwise interact physically and/or chemically, the remaining fluid can be flowed out of the fluid flow channel 94d. In another embodiment, the nanopore flow cell 12b that includes the structure 90 can be orientated so that fluid flow channels 92a, 92b, and 92c are disposed so that the reservoir 92 fills up from the bottom, where the bottom of the reservoir is the side where the fluid flow channels 92a, 92b, and 92c are disposed. Therefore, if the reservoir 92 is in fluid communication with a nanopore aperture, then the probability of the nanopore aperture being blocked partially or completely by an air bubble is reduced.

It should be emphasized that many variations and modifications may be made to the above-described embodiments. All such modifications and variations are intended to be included herein within the scope of this disclosure and protected by the following claims.

We claim at least the following:

1. A nanopore analysis system, comprising:
a nanopore flow cell including a cell reservoir, at least one fluid flow channel, an electrode, and a nanopore aperture, wherein the cell reservoir is in fluid communication with the fluid flow channel, wherein the nanopore aperture is in fluid communication with the cell reservoir, wherein the cell reservoir is in fluid communication with the electrode, and wherein the nanopore flow cell further comprises:
a first structure having the nanopore aperture;
a second structure adjacent the first structure, the second structure including a first opening that defines a portion of the cell reservoir and a second opening that defines a portion of the fluid flow channel;
a third structure adjacent the second structure, the third structure having the electrode disposed thereon and an opening for the fluid flow channel;
wherein a portion of the cell reservoir is defined on a first side by the first structure and another portion of the cell reservoir is defined on a second side by the third structure, wherein a portion of the fluid flow channel is defined on a first side by the first structure, and wherein a fluid can flow through the openings of the fluid flow channel into the fluid flow channels and into the cell reservoir.

2. The nanopore analysis system of claim 1, wherein at least one fluid flow channel is configured to flow fluid into the cell reservoir from the bottom of the cell reservoir.

3. The nanopore analysis system of claim 1, further comprising a spacer structure disposed between the second structure and the third structure, wherein the spacer structure includes an opening for the fluid flow channel and an opening for the cell reservoir.

4. The nanopore analysis system of claim 1, further comprising:
a first mixing reservoir structure positioned adjacent a backside of the third structure that is on the opposite side as the electrode, wherein the first mixing reservoir structure includes a mixing reservoir opening that defines a portion of a mixing reservoir and a mixing reservoir fluid channel opening that defines a portion of a mixing reservoir fluid flow channel; and
a second mixing reservoir structure positioned adjacent a backside of the first mixing reservoir structure that is on the opposite side as the third structure, wherein the second mixing reservoir structure includes a mixing reservoir fluid channel opening that defines a portion of a second mixing reservoir fluid flow channel,
wherein a portion of the mixing reservoir is defined on a front side by the backside of the third structure and on a backside by a front side of the second mixing reservoir structure.

5. The nanopore analysis system of claim 4, wherein the mixing reservoir is in fluid communication with the mixing reservoir fluid flow channel, and wherein the mixing reservoir fluid flow channel is in fluid communication with the fluid flow channel.

6. The nanopore analysis system of claim 4, wherein the mixing reservoir includes at least three fluid flow channels.

7. The nanopore analysis system of claim 1, wherein the nanopore flow cell includes at least three fluid flow channels.

8. A method for analyzing a polynucleotide, comprising:
providing a nanopore analysis system as described in claim 1;
introducing a target polynucleotide to the cell reservoir via the fluid flow channel;
applying a voltage gradient to the nanopore analysis system;
translocating the target polynucleotide through the nanopore aperture; and
monitoring the signal corresponding to the movement of the target polynucleotide with respect to the nanopore aperture.

9. The method of claim 8, wherein changes in the signal correspond to individual nucleotide monomers of the polynucleotide.

10. The method of claim 8, further comprising determining the nucleotide sequence of the target polynucleotide based on changes in the signal being monitored.
